(19) United States
(12) Patent Application Publication
(10) Pub. No.: US 2016/0287558 A1
(43) Pub. Date: Oct. 6, 2016

(54) ORAL PHARMACEUTICAL FORM FOR PREVENTING VASCULAR DISEASES, TABLET AS PHARMACEUTICAL FORM AND GELATIN CAPSULE AS PHARMACEUTICAL FORM

(71) Applicant: HYPERMARCAS S.A., São Paulo - Sp (BR)

(72) Inventors: Walker Magalhães LAHMANN, São Paulo (BR); Hilton Oliveira dos SANTOS FILHO, São Paulo (BR); Iara Silva Brauer MANTOVANI, Anápolis City (BR); Cristiano Martins VELOSO, Anápolis (BR); Simone Batista de OLIVEIRA, Anápolis (BR); Fabiana Lima CARVALHO, Anápolis (BR)

(21) Appl. No.: 15/035,404

(22) PCT Filed: Nov. 7, 2014
(86) PCT No.: PCT/BR2014/050009

§ 371 Date: May 9, 2016

(30) Foreign Application Priority Data
Nov. 8, 2013 (BR) 1020130288837

(51) Int. Cl. A61K 31/40; A61K 9/20; A61K 9/48; A61K 31/549; A61K 9/24; A61K 9/28; A61K 9/00; A61K 31/4178

(52) U.S. Cl. CPC A61K 31/40; A61K 9/0053; A61K 9/2009; A61K 31/4178; A61K 31/549; A61K 9/209; A61K 9/28; A61K 9/4825; A61K 9/485

(57) ABSTRACT

The present invention broadly relates to oral dosage forms for therapeutic use in the prevention of cardiovascular and cerebrovascular diseases in mammals, preferably in humans. Particularly, the invention relates to a tablet comprising (a) one or more HMG-CoA reductase inhibitors, (b) one or 5 more antagonists of angiotensin II receptors and (c) one or more thiazide diuretics.

### FIELD OF THE INVENTION

The present invention broadly relates to oral dosage forms, aimed at the prevention of cardiovascular and cerebrovascular diseases in mammals, particularly in humans. Particularly, the invention relates to pharmaceutical forms comprising (a) one or more HMG-CoA reductase inhibitors, (b) one or more antagonists of angiotensin II receptors and (c) one or more thiazide diuretics, where (a) is not in substantial physical contact with (b) or (c).

### BACKGROUND OF THE INVENTION

Although there are many formulas and strategies for pharmaceutical combinations or associations comprising many active principles, particularly in the prevention of cardiovascular and cerebrovascular diseases, there is a constant search for an effective, simple, inexpensive and safe association.

The fixed dose associations are more comfort to the patient, who ingests a single tablet, instead of three different units, thus also significantly reducing the cost of manufacturing, packaging, logistics and regulatory processes. It is estimated that a fixed dose combination of three active principles would reduce the final cost by up to 40% compared to the manufacturing cost of individual products.

However, fixed dose combinations or associations of this nature face difficulties in the production for certain pharmaceutical forms, such as tablets, especially related to:
a) Different solubilities;
b) Different dissolution profiles;
c) Incompatibility of excipients;
d) Different stability conditions;
e) Incompatibility of active principles.

In view of the difficulties above, a common problem is to maintain the performance that active principles have individually, when they are combined in the same pharmaceutical form, comprising multiple active principles and excipients.

According to the present invention, oral pharmaceutical forms were developed which comprise combinations of (a) HMG-CoA reductase inhibitors, particularly stabilized statins, (b) antagonists of the angiotensin II receptors particularly stabilized and (c) one or more thiazide diuretics that, surprisingly, provide performance of its components as obtained individually.

As employed herein, the “thiazide class” refers to benzothiadiazine structure, which comprises heterocyclic bicyclic compounds derived from benzene, with an heterocycle with 2 nitrogen atoms and one sulfur atom.

It was surprisingly found that a particular aspect to maintain the individual performance of the active principles that comprise the pharmaceutical form aspect of the invention is to prevent or minimize direct physical contact of HMG-CoA reductase inhibitors with the other two active principles, which are preferably mixed together.

Accordingly, a particularly preferred embodiment of the invention is a tablet in which the active principles are separated in two layers, one layer containing one or more HMG-CoA reductase inhibitors, and the other layer containing a mixture of one or more antagonists of angiotensin II receptors with one or more thiazide diuretics.

Optimal conditions obtained by the invention are understood as being the release of drugs, according to the dissolution profile of the reference active principles, i.e., of the individual compounds, as well as the robust and reproducible industrial production.

The combination of different active principles in the same pharmaceutical form is not an obvious procedure. For example, a significant compressive force is required for a bilayer tablet, so that the lower layer is sufficiently pressed by the upper layer, such process being substantially different from simple compression.

Thus, a specific formulation for oral solid dosage forms of active principles in combinations of fixed doses require specific formulations for bonding two or three layers, or to include multiparticulate forms (“pellets”) in capsules or tablets, or for pharmaceutical active principles on coating polymers.

The formulations prepared according to the present invention exhibit an in vitro behavior of active constituents similar to the performance achieved by the simple forms of individual products.

The isolated products are simple tablets, that is, pharmaceutical forms especially simple, when compared to pharmaceutical forms formed by the combination of active principles.

### BRIEF DESCRIPTION OF THE INVENTION

The pharmaceutical forms of the invention are directed to the prevention of cardiovascular and cerebrovascular diseases, particularly arterial hypertension, arterial coronary disease, acute coronary syndrome and stroke. The active principles of the preparation of the invention are discussed below.

HMG-CoA Reductase Inhibitors

Statins are known as HMG-CoA inhibitors (HMG-CoA is an acronym for “3-hydroxy-3-methylglutaryl-coenzyme A”), an enzyme of the liver tissue that produces mevalonate, a small molecule used in the synthesis of cholesterol and other mevalonate derivatives. Its inhibition reduces the amount of cholesterol produced which, in turn, reduces the total amount of LDL (“low density lipoprotein”) cholesterol. As high levels of LDL cholesterol are associated with morbidity and mortality from cardiovascular and cerebrovascular events, the effect of constant use of statins is particularly suitable to prevent such events.

It is known that statins have poor stability in acid medium, thus, particularly, the formulations proposed in the present invention have a matrix pH close to neutrality in order to minimize degradation, moreover, it has been also detected, in preliminary studies, that the use of antioxidants has an important role in molecule stabilization, for example, with the use of sodium metabisulfite, potassium metabisulfite, sodium sulfite, etc. The alkaline environment favors the dissolution of this active principle, thus enabling the adequate absorption by the gastrointestinal tract.

ARA—Antagonists of Angiotensin II Receptors

The class of angiotensin receptor antagonists (ARBs) is useful in the treatment of hypertension. For example, losartan, the first ARA made available on the market, blocks the AT1 angiotensin II receptors. It causes direct vasodilation, preventing the increase in aldosterone production by the adrenal glands, thus reducing the retention of sodium and water, thereby preventing hyperevolemia and consequent arterial hypertension.

Thiazide Diuretics

Diuretics lower blood pressure by reducing plasma volume and extracellular volume.

Thiazides were originally synthesized for being inhibitors of carbonic anhydrase (a class of diuretics that reduces HCO₃⁻ absorption and, indirectly, of Na⁺). However, thiazides (including hydrochlorothiazide) are very weak inhibitors of carbonic anhydrase. Particularly hydrochlorothiazide belongs to the group of salturetics and acts on the distal tubule of the nephron at the thiazide-sensitive cotransporter (TSC) level, which is a sodium-chloride symporter channel. Thus, there is an increased excretion of water and NaCI (by osmotic effect). This loss of water increases diuresis and decreases the extracellular fluid volume and, consequently, reduces blood pressure.

The ALLHAT American study (acronym of “Antihypertensive and Lipid-Lowering Treatment to Prevent Heart Attack Trial”, detailed at http://www.nhlbi.nih.gov/health/allhat/facts.htm), demonstrated that hydrochlorothiazide is as effective as other drugs and further prevents more cardiovascular complications than other drugs. This, associated with its low cost, which is an important factor in a therapy that can extend to the rest of life, makes it a first-line drug in managing hypertension.

The present invention aims at the combination of the three active principles in oral pharmaceutical forms, with a particular physical separation of active pharmaceutical principles, thus favoring aspects of stability, dissolution and compatibility among them.

### DESCRIPTION OF THE FIGURES

FIG. 1—Bilayer tablet, wherein one layer with one or two active pharmaceutical principles is compressed on the other layer, with one or two active principles, totaling three active principles.

FIG. 2—Trilayer tablet, wherein each layer with an active principle is compressed sequentially, totaling three layers and three active principles.

FIG. 3—Bilayer tablet containing multiunits (pellets) of an active pharmaceutical principle.

FIG. 4—Monoblock tablet containing multiunits (pellets) with one or two active pharmaceutical principles.

FIG. 5—Tablet with polymeric coating which includes one or two active pharmaceutical principles.

FIG. 6—Tablet included in the tablet, or “tablet in tablet”, in which the active principles are physically separated by two tablets.

FIG. 7—Hard gelatin capsule forms containing multiparticulate forms in pellets of three active principles.

FIG. 8—Dissolution profile of the atorvastatin obtained by the invention, in phosphate buffer medium, at pH 6.8, using apparatus 2 (paddle) rotating at 75 rpm, at 37° C. in 900 mL.

FIG. 9—Dissolution profile of the atorvastatin in phosphate buffer medium, at pH 6.8, using apparatus 2 (paddle) rotating at 75 rpm, at 37° C. in 900 mL—curve obtained with the Citalor 10 mg product containing only this active principle.

FIG. 10—Dissolution profile of hydrochlorothiazide obtained by the invention, in phosphate buffer medium, at pH 6.8, using apparatus 2 (paddle) rotating at 75 rpm, at 37° C. in 900 mL.

FIG. 11—Dissolution profile of hydrochlorothiazide in phosphate buffer medium, at pH 6.8, using apparatus 2 (paddle) rotating at 75 rpm, at 37° C. in 900 mL—curve obtained with Hyzaar, containing 50 mg of losartan+12.5 mg of hydrochlorothiazide.

FIG. 12—Dissolution profile of losartan obtained by the invention, in phosphate buffer medium, at pH 6.8, using apparatus 2 (paddle) rotating at 75 rpm, at 37° C. in 900 mL.

FIG. 13—Dissolution profile of losartan in phosphate buffer medium, at pH 6.8, using apparatus 2 (paddle) rotating at 75 rpm, at 37° C. in 900 mL - curve obtained with Hyzaar, containing 50 mg of losartan+12.5 mg of hydrochlorothiazide.

### DETAILED DESCRIPTION OF THE INVENTION

In a first aspect, the invention relates to pharmaceutical oral forms characterized by comprising (a) one or more HMG-CoA reductase inhibitors, particularly statins stabilized with antioxidants, (b) one or more angiotensin-II receptor antagonists and (c) one or more thiazide diuretics, wherein (a) is not in substantial physical contact with (b) or (c).

“Oral forms” are preferably solid oral forms, e.g., bilayer tablets, trilayer tablets, monoblock tablets including many active principles physically separated, coated tablets, tablets with additived coating with active principles and capsules containing multiparticulate forms, or pellets, of two or three active principles.

Without excluding any other, HMG-CoA reductase inhibitors suitable for the invention are one or more statins, particularly selected from atorvastatin, simvastatin, fluvastatin, lovastatin, mevastatin, pitavastatin, pravastatin, rosuvastatin, cerivastatin and pharmaceutically acceptable salts thereof. Atorvastatin, or pharmaceutically acceptable salts thereof, in crystalline or amorphous forms, are particularly used.

In a particular embodiment of the invention, said statins are stabilized by one or more antioxidants, for example, sodium metabisulfite, potassium metabisulfite, sodium sulfite, etc.

Without excluding any other, said suitable ARA or angiotensin II receptor antagonists of the invention are one or more of losartan, irbesartan, valsartan, olmesartan, telmisartan, azilsartan, eprosartan, candesartan and pharmaceutically acceptable salts thereof. Losartan, or pharmaceutically acceptable salts thereof, are particularly used.

Without excluding any other, suitable thiazide diuretics of the invention are one or more of cyclothiazide, trichlormethiazide, cyclopenthiazide, polythiazide, bendroflumethiazide, hydroflumethiazide, chlorothiazide, hydrochlorothiazide, benzothiazide, flumethiazide, methylcyclothiazide, butiazide and penflutizide. Hydrochlorothiazide, or pharmaceutically acceptable salt thereof, are particularly used.

A particular embodiment of the invention comprises a tablet which contains from 2 to 80 mg of atorvastatin, between 5 and 120 mg of losartan and between 5 and 75 mg of hydrochlorothiazide.

A particular oral solid preparation embodiment of the invention is a tablet containing a (a) granulate or simple mixture with excipients containing a HMG-CoA reductase inhibitor, particularly a stabilized statin, (b) a granulate or simple mixture with excipients containing an ARA, (c) granulate or with excipients of thiazide diuretic, wherein (a) is not in substantial physical contact with (b) or (c).

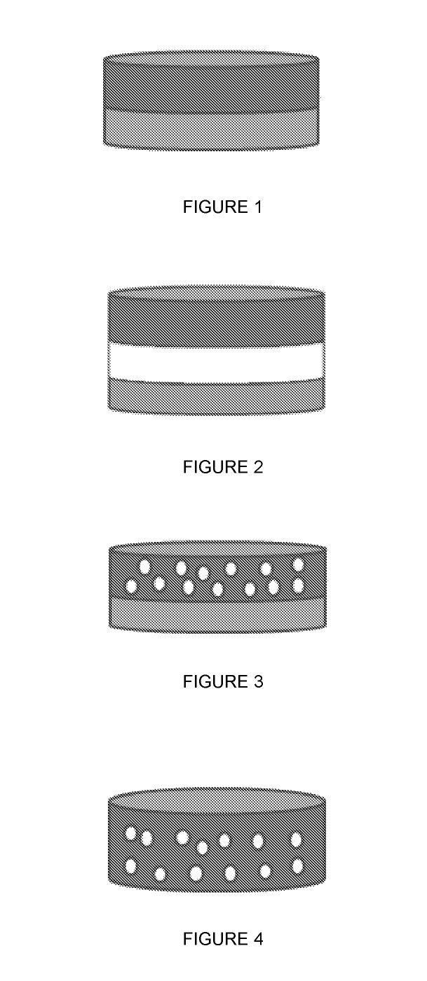

A particular embodiment of the invention is a tablet with two separate layers (as seen in FIG. 1), wherein a layer comprises formulated granules comprising one or more HMG-CoA reductase inhibitors (a), particularly stabilized atorvastatin, and the other layer is a combination of (b) a formulated granulate comprising one or more ARAs, particularly stabilized losartan, and (c) a solid solution granulate of one or more thiazide diuretics, particularly hydrochlorothiazide.

In another particular embodiment of the invention, the three active principles may be distributed in three different layers, constituting a trilayer tablet, as can be seen in FIG. 2.

Granulates of simple mixtures with excipients of the three essential components of the solid oral forms of the invention can be produced separately. Thus, the specific aspects related to the stability and solubility of each active principle are advantageously respected, enabling the best use of their features—i.e., the simple mixture of excipients to the thiazide diuretic, particularly hydrochlorothiazide, wherein the pH of the matrix close to neutrality benefits HMG-CoA reductase inhibitors sensitive to acidity, particularly to atorvastatin, and the intended addition of ARA, particularly losartan. Particularly, the pH of a granulate with HMG-CoA reductase inhibitors should be above 6.

In another preferred embodiment, the active principles can be combined into a monoblock tablet, with insertion of pellets or granules therein, as can be seen in FIGS. 3 and 4.

Pellets are understood as an agglomeration of fine powders containing one or more active substances and their respective excipient(s), in small spheroid units. These spheroidal units produced by spheronization or addition are independent units that do not necessarily contain the required entire dose of the active principle, but the set of pellets has the required dose.

Tablets containing pellets exhibit physical separation of active principles. In monoblock tablets, the active principles may be present in one or more different types of pellets immersed in the compressed mixture. The invention comprises, in one particular example, pellets comprising ARA and thiazide diuretics together.

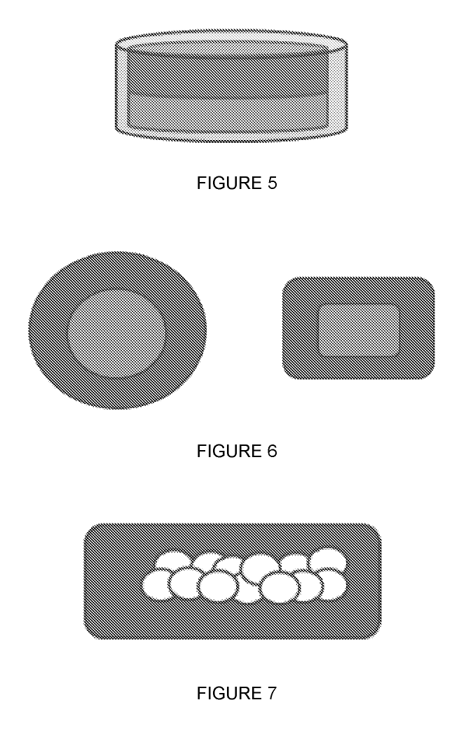

In other possible embodiments of the invention, monoblock or double layer tablets are produced, with or without pellets, with a coating comprising one or more active pharmaceutical principles. For example, one of the active principles is mixed with the coating material and sprinkled on the tablet, providing physical separation of the active principles, as can be seen in FIG. 5. Particular non-limiting examples of embodiments of this alternative are listed below:
a coating contains atorvastatin and coats a core that contains a mixture of losartan and hydrochlorothiazide;
a bilayer tablet, one with losartan and the other with hydrochlorothiazide, which has a coating comprising atorvastatin;
a coating containing a mixture of losartan and hydrochlorothiazide coats a core that contains atorvastatin;
a bilayer tablet, one with losartan and the other with atorvastatin, which has a coating comprising hydrochlorothiazide;
a bilayer tablet, one with hydrochlorothiazide and the other with atorvastatin, which has a coating comprising losartan;

In another particular embodiment, the tablets are formed in the “tablet-on-tablet” system, where a granulate or simple mixture is comprised in another tablet, as can be seen in FIG. 6.

In another particular embodiment, the active principles are combined into small tablets, powders or pellets, which fill gelatin capsules, preserving the physical separation of the active principles, as can be seen in FIG. 7.

### Tablets

Immediate or extended release coated tablets are solid pharmaceutical forms that may be orally administered, which can be produced by physical compression of the active principle and excipients, i.e. the set of substances which confer form, compressibility, mechanical resistance and modulate the release of the active principle. There is a core, which is the set formed by excipients and active ingredients, coated with a film that isolates the core and avoids contact with the atmospheric air, but which is soluble in the gastrointestinal environment.

The core can be produced by dry or wet granulometry. The granules can be produced, for example, with silicon dioxide, cellulose, for example, Avicel®, carbohydrates, starches and other ingredients. It may be mentioned as binders gelatin, alginate, cellulose ethers, e.g. CMC (carboxy-methyl-cellulose), (hydroxy-propyl-cellulose) and HPMC (hydroxy-propyl methyl cellulose), polyethylene glycols, ethylene oxide on polymerized form, polyvinylpyrrolidones and povidone. Detergent products may be used as sodium lauryl sulfate or dodecyl sulfate.

In the wet method, the mass formed, for example, by solid dispersion of the mixture of active ingredient, cellulose and colloidal silicon dioxide, is mixed in a rotating mill. Then, the dough is transferred into a granulator - for example, GLATT® by adding solvents, such as ethyl alcohol and povidone, until complete dispersion. The granules are dried by submitting the dough composed of active principle and excipients to fluidized-bed, followed by compression, which can be done in compressing machines, for example, rotary compressors.

The mass of the mixture, before compression, can receive spheronized pellets or be constructed by addition. The small spheres containing active principles can be actively mixed into the mass of excipients, while maintaining a uniform pellet distribution inside the monoblock or bilayer tablet.

Usual compression techniques are described, for example, in Remington: The Science and Practice of Pharmacy, 19th ed. Vol. 11 (1995) (Mack Publishing Co., Pennsylvania) and in Remington's Pharmaceutical Sciences, Chapter 89, pages 1633-1658 (Mack Publishing Company, 1990).

The compression can be done in rotary compressors, the shape of the cores (biplanar convex, cylindrical, oblong, etc.) and sizes can vary, in monoblock tablets, or in tablets with two or three layers.

The coat of a tablet aims to preserve stability of the active ingredient(s), by protection provided by core isolation from the external environment. Preferably, the coating is hydrophilic, permeable to water and to the moist environment of the gastrointestinal tract.

The materials which form coatings are, e.g. mixtures of polyvinylpyrrolidone or polyvinylpyrrolidone-polyvinyl acetate copolymer with hydroxypropylmethylcellulose (HPMC) or mixtures of cellulose derivatives, such as hydroxypropylmethylcellulose and ethylcellulose, or mixtures of polyvinyl alcohol/PEG/talc. These polymers are dispersed in ethanol or water to constitute the application vehicle. Pigments, talc or wetting agents can be added to the polymer solution in water or alcohol. Colorants and pigments may be also added, to provide a nice and different color to the pharmaceutical form, such as, for example, titanium dioxide and ferric oxide.

According to the invention, the tablet coating may contain active pharmaceutical principles. Thus, tablets with one or more active principles, separated into layers or with pellets, or granules produced separately, may be coated with polymers added with active pharmaceutical principles.

Particularly, the coating is a polymer, such as a polycarboxylic acid, for example, hydroxypropyl-methyl-cellulose phthalate, polyvinyl alcohol, polyvinyl-pyrrolidone or polymers known as “enteric polymers”, such as Eudragit L30D, dispersed in water with the addition of ammonia or other alkaline agent.

The coating forming material may be sprinkled on the tablet as an aqueous or alcoholic dispersion, using perforated bucket rotary coating equipments. Plasticizers can be added to the dispersion, such as triethyl acetate, other phthalates or polyethylene glycols.

The pharmaceutical forms of the present invention, particularly tablets, can contain a variety of known excipients, such as disintegrants, diluents, glidants and colorants. Among the disintegrants, those with a great expansion coefficient may be used, such as crospovidone, croscarmellose and starch glycolate. Diluents can be sucrose, dextrose, mannitol, sorbitol, starch, cellulose, lactose and other excipients known in the art.

Lubricants and glidants can be used in the production of tablets. Examples of lubricants and glidants can be hydrogenated vegetable oils, magnesium stearate, stearic acid, sodium lauryl sulfate, magnesium lauryl sulphate, colloidal silica, talc or mixtures of the excipients above. A preferred lubricant is magnesium stearate, or mixtures of magnesium stearate with colloidal silica.

The excipients used for compression are preferably binders, such as starches from different sources, for example, from maize, cellulose, such as Avicel®, silicon dioxide, such as Aerosil®, lactose, mannitol and polyethylene glycol with molecular weight between 400 and 6,000 Da, polyvinylpyrrolidone, such as Polyplasdone®, carboxymethyl cellulose, hypromellose, carboxymethyl starch, di-calcium phosphate, talc and lubricants, such as stearate. Various dyes, especially for differentiation between different dosages, may also be added.

In vitro Performance

Regarding the in vitro performance, the techniques of constructing tablets have a specific performance. One of the accepted ways of assessing the in vitro performance of tablets are the techniques of “dissolution” or the “dissolution profile”.

“Dissolution profile” means the determination of various concentrations of the drug in the dissolution medium, at different sampling points of time, in order to determine the curve of dissolved drug percentage as a function of time, in standardized apparatus, such as, for example, in the US Pharmacopoeia.

A drug orally administered can only be absorbed if it is dissolved in gastrointestinal media, which occurs only after its release from the pharmaceutical form.

The dissolution profile graphics are plots of the nominal percentage of the active pharmaceutical principle collected in the dissolution medium, i.e. the active pharmaceutical principle dissolved versus the time of each sampling.

The dissolution profiles have direct influence on the in vivo performance of pharmaceutical forms. In the theoretical limit, if there is no dissolution, or if the dissolution is not relevant, drug absorption does not occur and, therefore, it will not perform any biological effect. On the other hand, the dissolution indicates that an insoluble drug, treated by excipients, becomes soluble.

In addition to the features of drug solubility and the effect of excipients used, the construction of the pharmaceutical form has direct effect on the dissolution profile. A tablet with greater compressive force, for example, can retain the drug for longer periods of time, influencing the drug biological effect.

Thus, the dissolution profile is a typical expression of the drug in a particular pharmaceutical form, with direct impact on its clinical performance. One of the forms of measuring the drug dissolution in a pharmaceutical form is the “dissolution efficiency”, i.e. the area under the curve of the dissolution profile in relation to the total area of the plotted graph, in percentage.

A dissolution efficiency of 50% means that, during the whole experiment, the area under the curve of the dissolution profile represents 50% of the total area of the graph. In a hypothetical situation, if a tablet instantly dissolves 100% of the drug, the dissolution efficiency would be 100%. On the other hand, if a tablet does not release any percentage of the drug, it would be completely inefficient, i.e. the dissolution efficiency would be 0%.

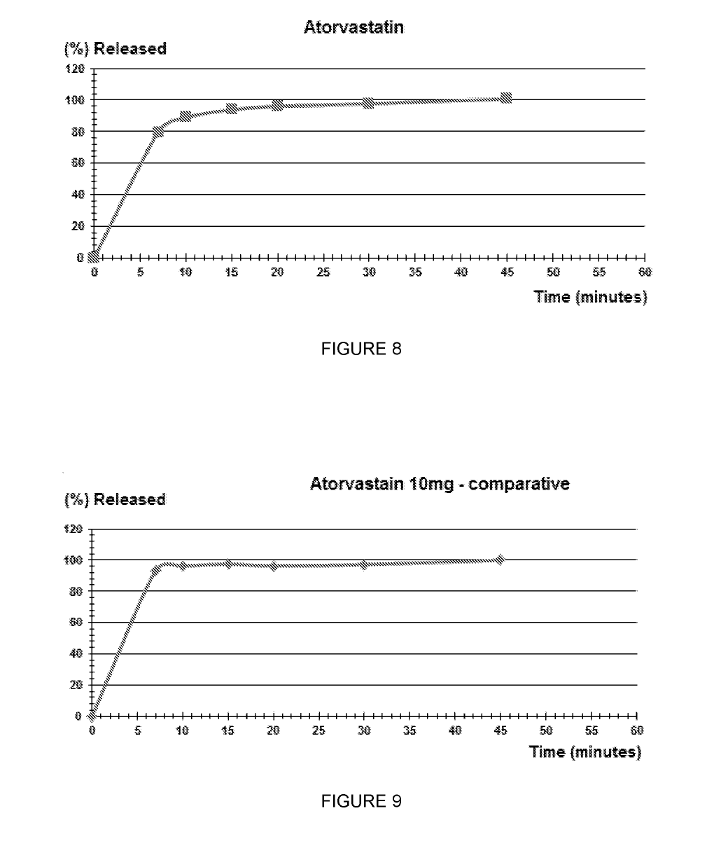

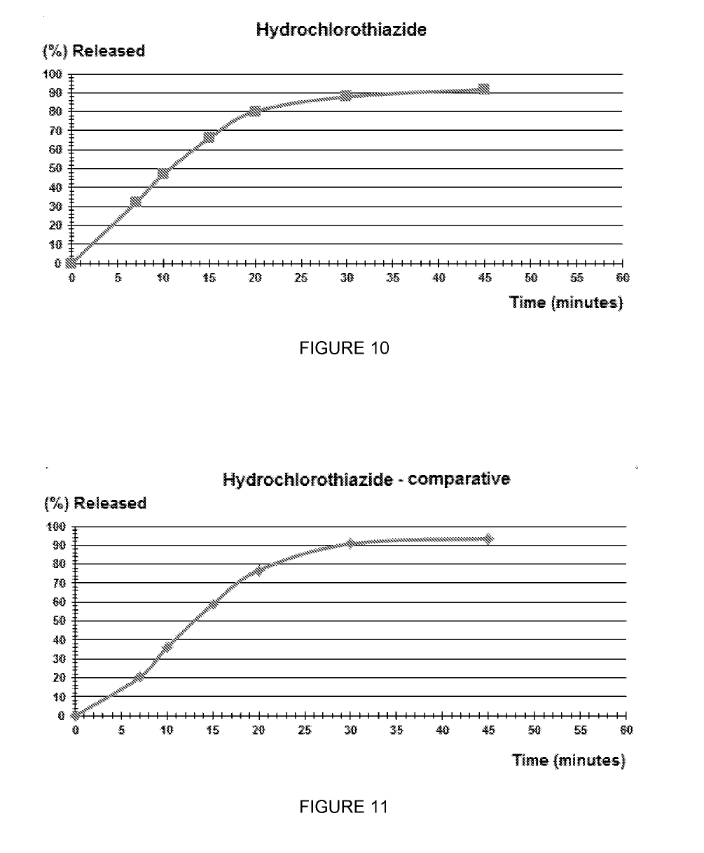

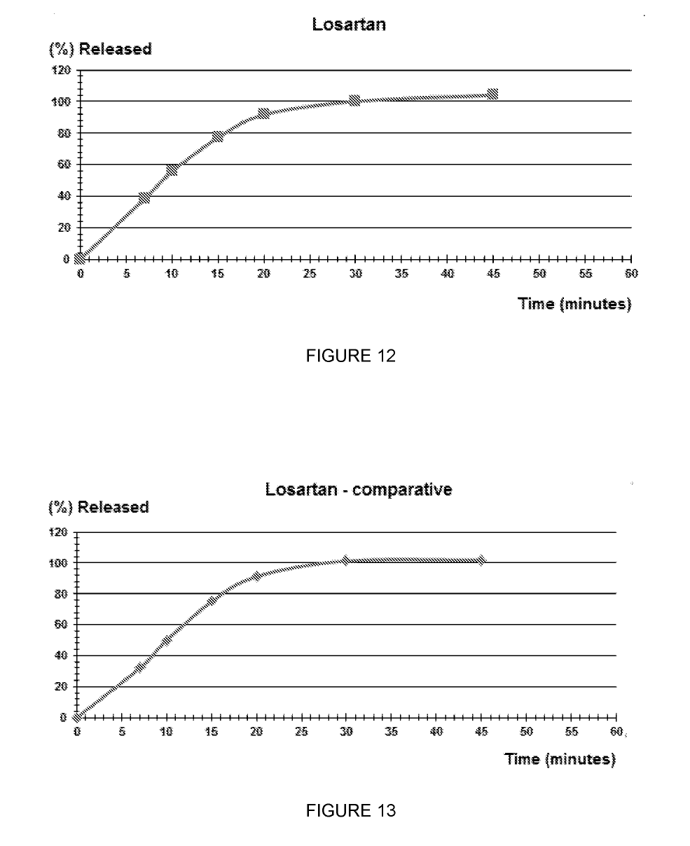

For in vivo behavior simulation, various means were tested in the study of the dissolution method (EMD), such as water, buffer, pH 6.8, hydrochloric acid, pH 1.2, and buffer, pH 4.5, and variables in methodology, such as apparatus, rotation and volume of medium were also tested, in order to meet the most possible detailed methodology. Particularly to the present invention, it was verified that 900 mL of buffer, pH 6.8, rotating at 75 rpm using apparatus 2 (paddle) at 37° C. is the most adequate dissolution methodology. The profiles presented in FIGS. 8 to 13, conducted according to the methodology described above, showed excellent in vitro correlation between medicines containing the isolated drugs commercially available and the fixed dose association of drugs of this invention, with approval at F2 (similarity factor), as recommended by the current standards.

The set comprising the core and the coating or the capsule provides stability to the active principles for a minimum period of two years. Stability can be defined as the absence of degradation within acceptable limits. Particularly, the stability studies of periods of six months were evaluated, which showed great stability, with all parameters meeting the predefined specifications, especially for the level of active principles which do not show significant variation and the impurities/degradation products within the limits set forth. It is known that a person skilled in the art, with the information and examples in this document, can carry out particular embodiments of the invention which are not expressly described, but which aim at the same function to achieve the same result, even though through a different manner. Such equivalent embodiments are covered by the attached set of claims.

### EXAMPLES

The following is an exemplary embodiment of this invention, which relates to bilayer coated tablets with separation of granules. Such example is merely illustrative of a particular embodiment of the invention, and for this reason, it does not represent any limitation beyond the restrictions set forth by set of claims below.

### Example 1

### Preparation According to the Present Invention

TABLE 1
LAYER 1

| Ingredient | Amount (mg) |
|---|---|
| Amorphous calcium atorvastatin | 10.36 |
| Cellulose | 89.02 |
| Lactose | 89.02 |
| Sodium metabisulfite | 1.60 |
| Crospovidone | 8 |
| Magnesium stearate | 2 |

Process

All items were passed through a sieve (except stearate) and mixed in a blender, in simple mixture. Then, sieved stearate was added and the mixture was blended.

LAYER 2

| Ingredient | Amount (mg) |
|---|---|
| Losartan potassium | 50 |
| Hydrochlorothiazide | 12.5 |
| Hydroxypropyl cellulose | 4.60 |
| Cellulose | 43.50 |
| Lactose | 62.20 |
| Pregelatinized corn starch | 24.88 |
| Yellow Dye | 0.40 |
| Magnesium stearate | 1.92 |

Process

Sieved losartan potassium, hydrochlorothiazide, hydroxypropyl cellulose, lactose, pregelatinized starch and cellulose, and the dye, separately sieved, are transferred to the mixer and homogenized. Magnesium stearate previously sieved was added to the mixer and mixed.

After bilayer compression of layers 1 and 2, the coating was conducted through a circular equipment with perforated bucket.

The coating used was a mixture of polyvinyl alcohol, macrogol and talc.

### Example 2

### In vitro Results

The components of the tablet described above were evaluated as for dissolution efficiency, as demonstrated in FIGS. 8 to 13.

The dissolution curves represent the expression of the release mechanism of active principles within the pharmaceutical form. Thus, it is possible to modulate the release to protect the active pharmaceutical principle, besides of improving the solubilization conditions. Excipients are used to optimize the behavior of the active pharmaceutical principle. In this invention, the active principles atorvastatin and hydrochlorothiazide are insoluble in water, and losartan is freely soluble.

The dissolution curves express, as demonstrated in FIGS. 8 to 13, the interaction between active principles and excipients—therefore, the dissolution efficiency is the significance of the pharmaceutical composition per se, and of the manufacturing process. As an example, hydrochlorothiazide alone, without the excipients, presents solubility close to zero.

It is verified that the composition object of this invention provides dissolution similar to the active principles, both in individual tablets and in combination.

### Example 3

### Clinical Study to Evaluate Pharmacokinetics in Healthy Subjects

The pharmaceutical form according to the present invention (containing atorvastatin, losartan and hydrochlorothiazide as active principles) is evaluated to verify if it has pharmacokinetic parameters comparable to medicines commercially available: Citalor® (Pfizer, containing only atorvastatin) and Hyzaar® (Merck Sharp & Dohme, containing losartan +hydrochlorothiazide) in healthy subjects.

The purpose of this clinical, randomized and cross-over study is to evaluate the pharmacokinetic profile of the pharmaceutical form, according to the present invention, by comparing the serum concentration of unchanged analytes (AT, LS and HCTZ) in healthy subjects.

The pharmacokinetic profile of the medicine is also evaluated by comparing the serum concentration of the following active metabolites:
1. O-hydroxy atorvastatin (2-hydroxy atorvastatin, O-HAT): atorvastatin metabolite
2. Carboxylic acid (E-3174, LS-CA): active metabolite of losartan

The criteria for inclusion of patients are:
1. voluntary participation;
2. age between 18 and 55 years, regardless of sex, clinically healthy and presenting normal laboratory parameters;
3. BMI 18.5 and 30.

The exclusion criteria are the following:
1. Participation in clinical trials in 12 months preceding the survey;
2. Clinical history of hypersensitivity reactions to atorvastatin, losartan, hydrochlorothiazide and/or any other component;
3. Presence of alterations in clinical and laboratory examinations that interfere in the study results;
4. Presence of lung, cardiovascular, neurological, endocrine, gastrointestinal or genitourinary diseases, or of other systems;
5. Acute disease within 7 days prior to the beginning of the study;
6. Chronic disease that determine medicine delivery, such as arterial hypertension, diabetes or any other that requires continuous use of any medicine, including the use of vitamins, mineral supplements and over-the-counter medicines;
7. Use of medicines that interact with any component of the pharmaceutical form, according to the present invention;
8. History of current use of tobacco, or within the last 12 months;
9. Current or past (less than 12 months) medical history of use of illicit drugs;

The calculated statistic sample was of 90 subjects, 30 on each arm. It is a prospective, randomized, open and crossed-over assay (3 sequences, 3 periods) controlled by the active comparative element.

Comparative treatment 1: Citalor (atorvastatin 10 mg);

Comparative treatment 2: Hyzaar® (losartan 50 mg+hydrochlorothiazide 12.5 mg);

Test treatment: pharmaceutical form according to the present invention containing atorvastatin 10 mg, losartan 50 mg and hydrochlorothiazide 12.5 mg.

Considering this as a cross-study, with 3 periods, each medicine is orally administered once (a medication in every study period).

The study also proposes the analysis of possible pharmacokinetic interactions between the analyzed medications, since interactions between these substances are not well understood.

The primary pharmacokinetic parameters, for comparability purposes, are the dosages of unchanged analytes (atorvastatin, losartan and hydrochlorothiazide), assessed by AUC, Tmax, Cmax and T1/2.

The secondary pharmacokinetic parameters are the dosages of active metabolites O-HAT and E-3174, assessed by AUC, Tmax, Cmax and T1/2.

The following are statistically evaluated:
Test treatment: atorvastatin, losartan and hydrochlorothiazide
Comparative treatment 1: atorvastatin,
Comparative treatment 2: losartan and hydrochlorothiazide.

In order to meet the objective of this study, three comparisons are required:
Test vs. comparative element 1 (evaluating the pharmacokinetics of atorvastatin);
Test vs. comparative element 2 (evaluating the pharmacokinetics of losartan);
Test vs. comparative element 2 (evaluating the pharmacokinetics of hydrochlorothiazide).

1-20. (canceled)

21. An oral pharmaceutical form for preventing vascular diseases, comprising:
(a) one or more HMG-CoA reductase inhibitors or pharmacologically acceptable salts thereof, stabilized with an antioxidant agent selected from one or more among sodium metabisulfite, potassium metabisulfite and sodium sulfite;
(b) one or more angiotensin II receptor antagonists, and
(c) one or more thiazide diuretics, or pharmacologically acceptable salts thereof, wherein (a) is not in substantial physical contact with (b) or (c).

22. The oral pharmaceutical form, according to claim 21, wherein (b) and (c) are mixed or associated with each other.

23. The oral pharmaceutical form, according to claim 21, wherein said HMG-CoA reductase inhibitors are one or more statins chosen among atorvastatin, simvastatin, fluvastatin, lovastatin, pravastatin, pitavastatin, mevastatin, rosuvastatin, cerivastatin and pharmaceutically acceptable salts thereof.

24. The oral pharmaceutical form, according to claim 21, wherein the statin is atorvastatin or pharmacologically acceptable salts thereof, in crystalline or amorphous forms.

25. The oral pharmaceutical form, according to claim 21, wherein said angiotensin II receptor antagonists are one or more among losartan, irbesartan, valsartan, olmesartan, telmisartan, azilsartan, eprosartan, candesartan and pharmaceutically acceptable salts thereof.

26. The oral pharmaceutical form, according to claim 25, wherein the antagonist of angiotensin II receptors is losartan, or pharmacologically acceptable salts thereof.

27. The oral pharmaceutical form, according to claim 21, wherein said thiazide diuretics are one or more from cyclothiazide, trichlormethiazide, cyclopenthiazide, polythiazide, bendroflumethiazide, hydroflumethiazide, chlorothiazide, hydrochlorothiazide, benzothiazide, flumethiazide, methylcyclothiazide, buthiazide, penfluthiazide and pharmaceutically acceptable salts thereof.

28. The oral pharmaceutical form, according to claim 27, wherein said diuretic is hydrochlorothiazide, or pharmacologically acceptable salts thereof.

29. The oral pharmaceutical form, according to claim 21, further comprising between 2 and 80 mg of atorvastatin, between 5 and 120 mg of losartan and between 5 and 75 mg of hydrochlorothiazide.

30. A tablet as a pharmaceutical form according to claim 21, having a second layer comprising losartan, hydrochlorothiazide and excipients.

31. The tablet, according to claim 30, having three layers, a first layer comprising atorvastatin and excipients, a second layer comprising losartan and excipients and a third layer comprising hydrochlorothiazide and excipients, in any order of compression.

32. The tablet, according to claim 30, wherein the tablet is a monoblock tablet with separation of granules, one of them comprising atorvastatin granules, the other comprising losartan granules and the third comprising hydrochlorothiazide granules.

33. The tablet, according to claim 30, further comprising multiparticulate units of atorvastatin with physical separation of atorvastatin in relation to hydrochlorothiazide and losartan and excipients.

34. The tablet, according to one of claim 30, wherein the tablet is a coated tablet.

35. The tablet, according to claim 34, wherein the coating contains atorvastatin.

36. The tablet, according to claim 34, wherein the coating contains losartan and/or hydrochlorothiazide.

37. The tablet, according to claim 30, wherein the tablet provides dissolution efficiency for atorvastatin, releasing ≧80% in 15 minutes; losartan, releasing ≧85% in 30 minutes; and hydrochlorothiazide releasing ≧75% in 30 minutes; in phosphate buffer, pH 6.8, using the United States Pharmacopeia dissolution apparatus 2 method and rotating at 75 rpm, at 37 degrees, in 900 mL.

38. A gelatin capsule as pharmaceutical form, according to claim 21, containing pellets of the active principles (a), (b) and (c), and pharmaceutically acceptable excipients.
